(19) United States
(12) Patent Application Publication
(10) Pub. No.: US 2023/0144374 A1
(43) Pub. Date: May 11, 2023

(54) MACHINE LEARNING MODELS FOR GENOMIC PREDICTIVE DATA ANALYSIS

(71) Applicant: Optum, Inc., Minnetonka, MN (US)

(72) Inventors: Rakhee Srivastava, Delhi (IN); Vivek Kumar Yadav, Lucknow (IN); Pranav Mahajan, Ghaziabad (IN); Lalit Singla, Gurugram (IN)

(21) Appl. No.: 17/453,705

(22) Filed: Nov. 5, 2021

(51) Int. Cl. G16B 5/20; G06N 3/08; G16B 40/00

(52) U.S. Cl. CPC G16B 5/20; G06N 3/086; G16B 40/00

(57) ABSTRACT

Various embodiments of the present invention provide methods, apparatus, systems, computing devices, computing entities, and/or the like for performing genomic predictive data analysis operations. For example, certain embodiments of the present invention utilize systems, methods, and computer program products that perform genomic predictive data analysis operations by using at least one of viral genomic processing machine learning models and bacterial genomic processing machine learning models.

### BACKGROUND

Various embodiments of the present invention address technical challenges related to performing genomic predictive data analysis operations and address the efficiency and reliability shortcomings of existing genomic predictive data analysis solutions.

### BRIEF SUMMARY

In general, embodiments of the present invention provide methods, apparatus, systems, computing devices, computing entities, and/or the like for perform genomic predictive data analysis operations. For example, certain embodiments of the present invention utilize systems, methods, and computer program products that perform genomic predictive data analysis operations by using at least one of viral genomic processing machine learning models and bacterial genomic processing machine learning models.

In accordance with one aspect, a method is provided. In one embodiment, the method comprises: identifying a first genomic sequence set; for each first genomic sequence in the first genomic sequence set: determining, using a frequency-based k-mer extraction layer of a viral genome processing machine learning model and based at least in part on the first genomic sequence, one or more frequent k-mers of the first genomic sequence; and determining, using a one-dimensional convolutional neural network layer of the viral genome processing machine learning model and based at least in part on the frequency-based refined representation, a viral replication origin k-mer of the one or more frequent k-mers; and performing one or more prediction-based actions based at least in part on each viral replication origin k-mer.

In accordance with another aspect, a computer program product is provided. The computer program product may comprise at least one computer-readable storage medium having computer-readable program code portions stored therein, the computer-readable program code portions comprising executable portions configured to: identify a first genomic sequence set; for each first genomic sequence in the first genomic sequence set: determine, using a frequency-based k-mer extraction layer of a viral genome processing machine learning model and based at least in part on the first genomic sequence, one or more frequent k-mers of the first genomic sequence; and determine, using a one-dimensional convolutional neural network layer of the viral genome processing machine learning model and based at least in part on the frequency-based refined representation, a viral replication origin k-mer of the one or more frequent k-mers; and perform one or more prediction-based actions based at least in part on each viral replication origin k-mer.

In accordance with yet another aspect, an apparatus comprising at least one processor and at least one memory including computer program code is provided. In one embodiment, the at least one memory and the computer program code may be configured to, with the processor, cause the apparatus to: identify a first genomic sequence set; for each first genomic sequence in the first genomic sequence set: determine, using a frequency-based k-mer extraction layer of a viral genome processing machine learning model and based at least in part on the first genomic sequence, one or more frequent k-mers of the first genomic sequence; and determine, using a one-dimensional convolutional neural network layer of the viral genome processing machine learning model and based at least in part on the frequency-based refined representation, a viral replication origin k-mer of the one or more frequent k-mers; and perform one or more prediction-based actions based at least in part on each viral replication origin k-mer.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of an architecture that can be used to practice embodiments of the present invention.

FIG. 2 provides an example predictive data analysis computing entity in accordance with some embodiments discussed herein.

FIG. 3 provides an example client computing entity in accordance with some embodiments discussed herein.

FIG. 4 is a data flow diagram of an example process for performing predictive viral genomic analysis on a genomic sequence set in accordance with some embodiments discussed herein.

FIG. 5 provides an operational example of a viral genomic processing machine learning model in accordance with some embodiments discussed herein.

FIG. 6 is a flowchart diagram of an example process for generating a viral strain prediction for an input viral genomic sequence in accordance with some embodiments discussed herein.

FIG. 7 is a data flow diagram of an example process for performing bacterial predictive genomic analysis on a genomic sequence set in accordance with some embodiments discussed herein.

FIG. 8 is a flowchart diagram of an example process for performing one or more prediction-based actions based at least in part on a set of predicted resistant bacterial segments in accordance with some embodiments discussed herein.

### DETAILED DESCRIPTION

Various embodiments of the present invention now will be described more fully hereinafter with reference to the accompanying drawings, in which some, but not all, embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense unless otherwise indicated. The terms “illustrative” and “exemplary” are used to be examples with no indication of quality level. Like numbers refer to like elements throughout. Moreover, while certain embodiments of the present invention are described with reference to genomic predictive data analysis, one of ordinary skill in the art will recognize that the disclosed concepts can be used to perform other types of data analysis tasks.

### I. Overview and Technical Improvements

Various embodiments of the present invention improve efficiency of performing genomic predictive data analysis operations by performing feature extraction on selected defined-length subsequences of genomic sequences known as k-mers. For example, in one embodiment, a proposed solution determines, using the processor and a frequency-based k-mer extraction layer of a viral genome processing machine learning model, and based at least in part on the first genomic sequence, one or more frequent k-mers of the first genomic sequence; and then determines, using the processor and a one-dimensional convolutional neural network layer of the viral genome processing machine learning model, and based at least in part on the frequency-based refined representation, a viral replication origin k-mer of the one or more frequent k-mers. Performing genomic predictive data analysis operations based at least in part on k-mer features replaces the need for performing complex and computationally expensive feature extraction operations on typically long and character-intensive genomic sequences with less complex operations needed to detect selected k-mers such as frequent k-mers. In this way, various embodiments of the present invention reduce the number of processing operations needed to perform genomic predictive data analysis operations, make important technical contributions to improving efficiency of performing genomic predictive data analysis operations, and substantially improve the field of genomic predictive data analysis.

Various embodiments of the present invention relate to identifying and processing gene segments of interest, for example viral gene segments of interest or bacterial gene segments of interest. In some embodiments, a proposed framework processes a gene sequence to identify k-mers, then processes the k-mers to identify which gene segments may be drug resistant, and then processes drug resistant gene segments to determine alternative treatment recommendations for the identified drug resistant gene segments.

In some embodiments, a proposed system targets the genes encoding a viral replication machinery by using inhibitor enzymes/molecules. In some embodiments, a proposed systems detects the presence of antibiotic resistance in a bacterial strain. In some embodiments, by starting from sequence alignment, and by using isolated and known antibiotic resistant gene segments as probes against the sample bacterial strains, the system is able to identify a resistant strain. With the data gathered over a period of time, the system trains models to identify resistance patterns in order to proactively identify resistant strains with high accuracy without the need for gene probes. This solution also helps in clinical decision-making by utilizing recommender systems that identify alternate route of treatments and alternate antibiotics, along with their respective efficacies. These results can then be utilized on a global scale to identify resistance hotspots across geographies; perform trend analysis with respect to antibiotics resistance, most vulnerable strains, most common antibiotics gaining resistance, and the like; and generate recommendations for the medical community to take timely corrective measures and spread information via Fast Healthcare Interoperability Resources (FHIR) resources.

In some embodiments, a proposed system includes a viral genome replication machinery identification engine that is configured to prevent retroviruses from replicating by correctly identifying the origin of replication inside the retroviral genome. This aims to assist scientists to come up with the right inhibitors or effective chemical/biological agents to block the viral replication points and render the virus ineffective. In some embodiments, a proposed system includes a bacterial genome antibiotic resistance identification engine that is configured to store antibiotic resistance gene segments and use those gene segments as marker probes to align the identified gene segments against sample bacterial genomes. The noted alignment may be used to identify potential matches in the sample genomes and to detect the presence of antibiotic resistance gene segments in a sample bacterial genome.

In some embodiments, a proposed system can accurately identify the origin of replication of viral strains and take counter measures to inhibit that stretch of nucleic acids to initiate the replication process. In doing so, the proposed system can ultimately contain the viral spread and lower the symptomatic and non-symptomatic health impact for those who are infected. The system can also help scientists refine the relevant genomics research to come up with the right inhibitors, based at least in part on the identified segments for more effective results on viral containment, by using chemical/biological agents.

### II. Definitions

The term “genomic sequence set” may be a data construct that is configured to describe a set of one or more genomic sequences that are extracted from a repository of genomic sequence data. In some embodiments, the genomic sequence data is received/retrieved/extracted from one or more genomic sequence databases, such as the National Center for Biotechnology Information (NCBI) genomic sequence database, the European Molecular Biology Laboratory (EMBL) genomic sequence database, DNA Data Bank of Japan (DDBJ) genomic sequence database, Protein Data Bank (PDB) genomic sequence database, Optum® genomic sequence database, and/or the like. In some embodiments, the genomic sequence set that is provided as an input for performing predictive viral genomic analysis comprises genomic sequences that are detected to have viral infections. In some embodiments, the genomic sequence set that is provided as an input for performing predictive viral genomic analysis comprises genomic sequences that are suspected of having viral infections. In some embodiments, the genomic sequence set that is provided as an input for performing bacterial predictive genomic analysis comprises genomic sequences that are suspected of having bacterial infections.

The term “viral replication origin k-mer” may be a data construct that is configured to describe a k-mer of a genomic sequence (i.e., a k-length subsequence of the genomic sequence) that is computationally determined/predicted to be a source of replication of a virus that is associated with the genomic sequence. The viral replication origin k-mer of a genomic sequence may in some embodiments be a detected/frequent k-mer of the genomic sequence that is determined to be the most likely source of a viral infection that is associated with the noted genomic sequence. In some embodiments, the viral replication origin of a genomic sequence is a frequent k-mer of the genomic sequence that has a highest likelihood output as generated by the one-dimensional convolutional layer of a viral genome processing machine learning model. In some embodiments, if two or more frequent k-mers of a genomic sequence have an equal output likelihood as generated by the one-dimensional convolutional layer of a viral genome processing machine learning model, then the viral replication origin of a genomic sequence may be selected from the noted two or more frequent k-mers based at least in part on defined selection criteria (for example by selecting the frequent k-mers of the noted two or more frequent k-mers that has a lower character length). In some embodiments, if two or more frequent k-mers of a genomic sequence have an equal output likelihood as generated by the one-dimensional convolutional layer of a viral genome processing machine learning model, then a combination of the noted two or more frequent k-mers is deemed to be viral replication origins of the corresponding genomic sequence.

The term “viral genome processing machine learning model” may be a data construct that is configured to describe parameters, hyper-parameters, and/or defined operations of a machine learning model that is configured to process a genomic sequence in order to generate a predictive output for the genomic sequence based at least in part on one or more k-mers of the genomic sequence. In some embodiments, the viral genome processing machine learning model comprises at least a k-mer extraction layer that is configured to determine one or more k-mers of a genomic sequence (e.g., one or more frequent k-mers of the genomic sequence) and a one-dimensional convolutional neural network layer that is configured to process the one or more k-mers to determine a predictive output for the genomic sequence (e.g., a viral replication origin k-mer for the genomic sequence). In some embodiments, the frequency-based k-mer extraction layer of a viral genome processing machine learning model is configured to process a genomic sequence to determine one or more frequent k-mers of the genomic sequence. In some embodiments, the one-dimensional convolutional layer of a viral genome processing machine learning model is configured to process an input k-mer (e.g., an input frequent k-mer) in order to determine an output value that describes a likelihood that the input k-mer is a viral replication origin k-mer for a genomic sequence that is associated with the noted input k-mer. In some embodiments, a frequent k-mer of a genomic sequence is determined to be the viral replication origin k-mer if the output likelihood that is generated for the frequent k-mer by the one-dimensional convolutional layer is higher than the output likelihoods generated by the one-dimensional convolutional layer for other frequent k-mers of the noted genomic sequence. For example, if a genomic sequence is associated with three frequent k-mers k₁, k₂, and k₃ that are respectively associated with output likelihood values p₁, p₂, and p₃, if p₁>p₂>p₃, then k₁ may be selected as the viral replication origin k-mer for the genomic sequence. In some embodiments, inputs to a viral genome processing machine learning model comprise a vector for a genomic sequence (e.g., a one-hot-coded vector of the genomic sequence) and/or a matrix for a genomic sequence set (e.g., a matrix describing the one-hot-coded vector of each genomic sequence). In some embodiments, outputs of a viral genome processing machine learning model comprise a vector describing the target viral replication origin k-mer of a particular genomic sequence.

The term “frequent k-mer” may be a data construct that is configured to describe a k-mer whose occurrence frequency score satisfies (e.g., exceeds) an occurrence frequency score threshold, where the occurrence frequency score of a k-mer with respect to a genomic sequence is detected based at least in part on a count of occurrence of the k-mer within the character string described by the noted genomic sequence. In some embodiments, the occurrence frequency score threshold that is used to classify a detected k-mer as a frequent k-mer or as an infrequent k-mer is determined based at least in part on an occurrence frequency score of a hypothesis k-mer that is determined to be a likely viral replication origin k-mer based at least in part on preexisting data about the virus at issue. In some embodiments, the frequency-based k-mer extraction layer of a viral genome processing machine learning model is configured to process a genomic sequence to determine one or more frequent k-mers of the genomic sequence. In some embodiments, a k-mer of a genomic sequence is any subsequence of the genomic subsequence that has a character length of k, where k may be determined by an optimal k-mer range for the genomic subsequence. In some embodiments, determining the one or more frequent k-mers of a particular first genomic sequence comprises determining one or more detected k-mers of the particular first genomic sequence; for each detected k-mer, determining an occurrence frequency score within the particular first genomic sequence; and determining the one or more frequent k-mers based at least in part on each occurrence frequency score. In some embodiments, determining the one or more frequent k-mers based at least in part on each occurrence frequency score comprises: identifying a hypothesis k-mer of the one or more frequent k-mers; and for each detected k-mer, determining that the detected k-mer is one of the one or more frequent k-mers if the occurrence frequency score for the detected k-mer exceeds the occurrence frequency score for the hypothesis k-mer.

The term “frequency-based k-mer extraction layer” of a viral genome processing machine learning model may be a data construct that is configured to describe a set of computer-implemented operations that are configured to process a genomic sequence to determine one or more frequent k-mers of the genomic sequence. In some embodiments, a k-mer of a genomic sequence is any subsequence of the genomic subsequence that has a character length of k, where k may be determined by an optimal k-mer range for the genomic subsequence. In some embodiments, a frequent k-mer is a k-mer whose occurrence frequency score satisfies (e.g., exceeds) an occurrence frequency score threshold, where the occurrence frequency score of a k-mer with respect to a genomic sequence is detected based at least in part on a count of occurrence of the k-mer within the character string described by the noted genomic sequence. In some embodiments, the occurrence frequency score threshold that is used to classify a detected k-mer as a frequent k-mer or as an infrequent k-mer is determined based at least in part on an occurrence frequency score of a hypothesis k-mer that is determined to be a likely viral replication origin k-mer based at least in part on preexisting data about the virus at issue. In some embodiments, a frequency-based k-mer extraction layer is configured to process a genomic sequence using a frequent words algorithm to determine one or more frequent k-mers of the genomic sequence.

The term “one-dimensional convolutional neural network layer” of a viral genome processing machine learning model may be a data construct that is configured to describe a set of computer-implemented operations that are configured to process an input k-mer (e.g., an input frequent k-mer) in order to determine an output value that describes a likelihood that the input k-mer is a viral replication origin k-mer for a genomic sequence that is associated with the noted input k-mer. In some embodiments, a frequent k-mer of a genomic sequence is determined to be the viral replication origin k-mer if the output likelihood that is generated for the frequent k-mer by the one-dimensional convolutional layer is higher than the output likelihoods generated by the one-dimensional convolutional layer for other frequent k-mers of the noted genomic sequence. For example, if a genomic sequence is associated with three frequent k-mers k₁, k₂, and k₃ that are respectively associated with output likelihood values p₁, p₂, and p₃, if p₁>p₂>p₃, then k₁ may be selected as the viral replication origin k-mer for the genomic sequence. In some embodiments, the one-dimensional convolutional layer of a viral genome processing machine learning model is associated with a stride value and/or a kernel size. In some embodiments, the stride value and/or the kernel size of a one-dimensional convolutional layer defines the character length of input k-mers that may be provided as inputs to the one-dimensional convolutional layer. For example, in some embodiments, processing a frequent k-mer using a one-dimensional convolutional layer first comprises selecting the one-dimensional convolutional layer from a set of candidate one-dimensional convolutional layers each associated with a stride value and/or kernel size, where the selection of the one-dimensional convolutional layer comprises selecting a candidate one-dimensional convolutional layer whose stride value and/or kernel size is a defined ratio of the character length of the frequent k-mer. For example, in an exemplary embodiment, if the character length of a frequent k-mer is 100, then the one-dimensional convolutional layer should have a kernel size of [18, 22].

The term “viral genome cluster” may be a data construct that is configured to describe a subset of a plurality of viral replication origin k-mers that is deemed to have similar viral/genomic properties. In some embodiments, determining the viral genome clusters comprises: (i) mapping each viral replication origin k-mer to an n-dimensional value in an n-dimensional space, and (ii) performing one or more clustering operations (e.g., one or more k-means clustering operations, one or more k-nearest-neighbor clustering operations, and/or the like) on the n-dimensional space to generate the viral genome cluster. Examples of features that may be associated with the n dimensions of the n-dimensional space comprise the number of adenine (A) characters in a k-mer, the number of thymine (T) characters in a k-mer, the number of guanine (G) characters in a k-mer, the number of cytosine (C) characters in a k-mer, the ratio of adenine (A) characters in a k-mer, the ratio of thymine (T) characters in a k-mer, the ratio of guanine (G) characters in a k-mer, the ratio of cytosine (C) characters in a k-mer, features describing the number and/or the ratio of combinations of the noted genomic base characters in a k-mer, features describing the number and/or the ratio of genomic base characters in a specified ratio of a k-mer (e.g., in a substring of the k-mer that includes the first two percent of the genomic base characters of the k-mer), features describing the number and/or the ratio of combinations of genomic base characters in a specified ratio of a k-mer (e.g., in a substring of the k-mer that includes the first two percent of the genomic base characters of the k-mer), and/or the like.

The term “viral cluster similarity measure” may be a data construct that is configured to describe a deviation measure for an input viral genomic sequence with respect to a viral genome cluster. For example, in some embodiments, to determine a viral cluster similarity measure for a viral genome cluster, a genomic analysis engine may: (i) identify one or more selected k-mers (e.g., one or more defined k-mers) of the input viral genomic sequence, (ii) map each selected k-mer to the multi-dimensional space of the viral genome cluster, (iii) for each selected k-mer, determine a distance measure between the mapping of the k-mer in the multi-dimensional space and a defined point of the viral genome cluster in the multi-dimensional space (e.g., a defined centroid point of the viral genome cluster in the multi-dimensional space), and (iv) determine the viral cluster similarity measure based at least in part on each distance measure for a selected k-mer (e.g., based at least in part on a sum of each distance measure for a selected k-mer, based at least in part on a statistical distribution measure such as an average of each distance measure for a selected k-mer, and/or the like). As another example, in some embodiments, to determine a viral cluster similarity measure for a viral genome cluster, a genomic analysis engine may: (i) identify one or more selected k-mers (e.g., one or more defined k-mers) of the input viral genomic sequence, (ii) map each selected k-mer to the multi-dimensional space of the viral genome cluster, (iii) process each selected k-mer using a one-dimensional convolutional layer of a viral genome processing machine learning model to generate an output likelihood for the selected k-mer, (iv) for each selected k-mer, determine a distance measure between the mapping of the k-mer in the multi-dimensional space and a defined point of the viral genome cluster in the multi-dimensional space (e.g., a defined centroid point of the viral genome cluster in the multi-dimensional space), (v) for each selected k-mer, determine a weighted distance based at least in part on the distance measure for the selected k-mer and the generated output likelihood for the selected k-mer (e.g., based at least in part on the output of applying the distance measure for the selected k-mer to the generated output likelihood for the selected k-mer), and (iv) determine the viral cluster similarity measure based at least in part on each weighted distance measure for a selected k-mer (e.g., based at least in part on a sum of each weighted distance measure for a selected k-mer, based at least in part on a statistical distribution measure such as an average of each weighted distance measure for a selected k-mer, and/or the like).

The term “viral strain prediction” may be a data construct that is configured to describe one or more viral/genomic properties of a viral infection that is associated with an input viral genomic sequence (e.g., where the viral infection may be of a novel viral strand). For example, in some embodiments, a genomic analysis engine may determine that the input viral genomic sequence has a viral strain prediction defined by one or more viral/genomic properties of a viral genome cluster having a highest viral cluster similarity measure among the viral cluster similarity measures of the plurality of viral cluster similarity measures. As another example, in some embodiments, a genomic analysis engine may determine that the input viral genomic sequence has a viral strain prediction defined by one or more viral/genomic properties of each viral genome cluster having a threshold-satisfying viral cluster similarity measure.

The term “genome processing machine learning model” may be a data construct that is configured to describe parameters, hyper-parameters, and/or defined operations of a machine learning model that is configured to apply a two-dimensional convolution to a two-dimensional representation of genomic sequence set (e.g., a two-dimensional data object including each one-hot-coded representation of a genomic sequence of the genomic sequence set) to determine each segment of the two-dimensional representation that is likely to be associated with a resistant bacterial segment. In some embodiments, in addition to and/or instead of using the two-dimensional convolutional operations, the genome processing machine learning model is configured to process the genomic sequence set using other pattern matching operations in order to determine each segment of the genomic sequence set that is likely to be associated with a resistant bacterial segment.

The term “resistant bacterial segment” may be a data construct that is configured to describe a segment of a genomic sequence set that is determined to be resistant to a set of one or more antibacterial medications. In some embodiments, a bacterial genome processing machine learning model processes a genomic sequence set to determine one or more resistant bacterial segments of the genomic sequence set. In some embodiments, a resistant bacterial segment is a two-dimensional subset of a genomic sequence. In some embodiments, to determine a resistant bacterial segment for a genomic sequence, a bacterial genome processing machine learning model utilizes a two-dimensional convolutional neural network layer, such as a two-dimensional convolutional neural network layer whose stride value determined based at least in part on a defined ratio of a number of elements of the genomic sequence set, or a two-dimensional convolutional neural network layer whose kernel dimension lengths are determined based at least in part on a defined ratio of the dimension lengths of the two dimensions of the genomic sequence. In some embodiments, the bacterial genome processing machine learning model is further configured to determine a non-resistant antibacterial recommendation for each resistant bacterial segment that describes an antibacterial medication to which the resistant bacterial segment is deemed to be non-resistant. In some embodiments, for each non-resistant antibacterial recommendation for a genomic sequence set, the bacterial genome processing machine learning model generates a recommendation score that describes an inferred/computed likelihood that the noted genomic sequence is non-resistant to an antibacterial medication of the non-resistant antibacterial recommendation.

### III. Computer Program Products, Methods, and Computing Entities

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatus, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations. Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. Exemplary System Architecture

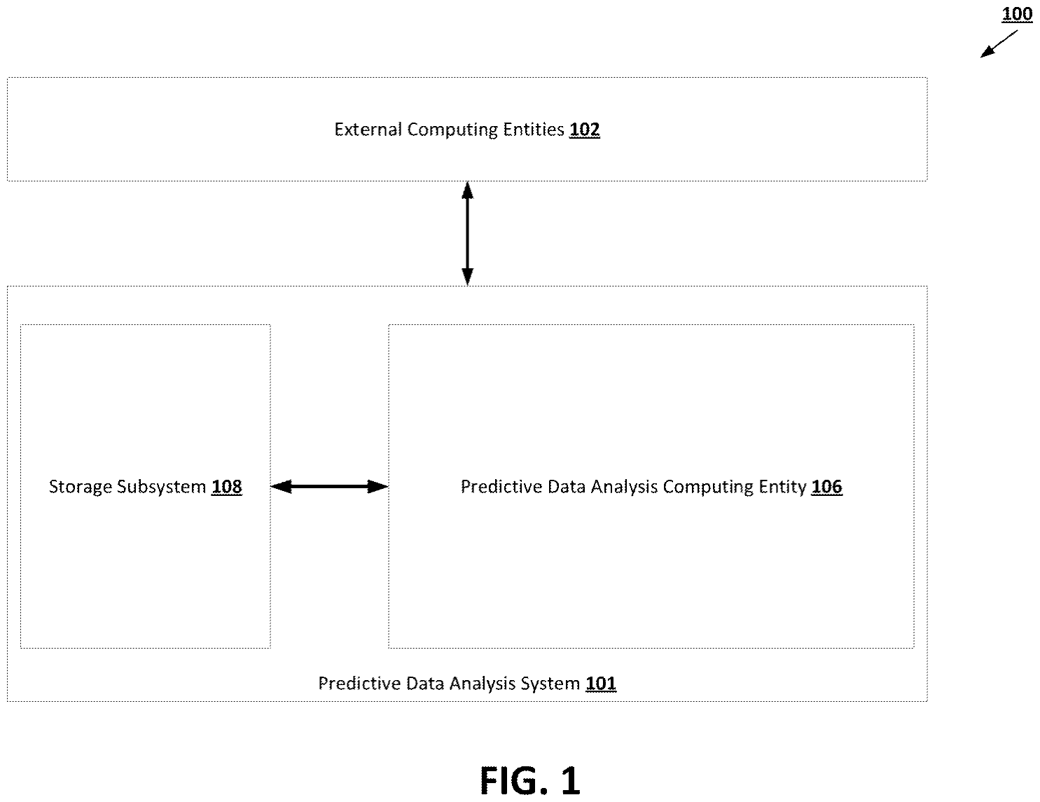

FIG. 1 is a schematic diagram of an example architecture 100 for performing genomic predictive data analysis. The architecture 100 includes a predictive data analysis system 101 configured to receive genomic predictive data analysis requests from client computing entities 102, process the genomic predictive data analysis requests to generate predictions, provide the generated predictions to the client computing entities 102, and automatically perform prediction-based actions based at least in part on the generated predictions. An example of a prediction-based action that can be performed using the predictive data analysis system 101 is identifying a viral replication origin segment of a genomic sequence and/or an antibacterial resistant segment of a genomic sequence.

In some embodiments, predictive data analysis system 101 may communicate with at least one of the client computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The predictive data analysis system 101 may include a predictive data analysis computing entity 106 and a storage subsystem 108. The predictive data analysis computing entity 106 may be configured to receive genomic predictive data analysis requests from one or more client computing entities 102, process the genomic predictive data analysis requests to generate predictions corresponding to the genomic predictive data analysis requests, provide the generated predictions to the client computing entities 102, and automatically perform prediction-based actions based at least in part on the generated predictions.

The storage subsystem 108 may be configured to store input data used by the predictive data analysis computing entity 106 to perform genomic predictive data analysis as well as model definition data used by the predictive data analysis computing entity 106 to perform various genomic predictive data analysis tasks. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Predictive Data Analysis Computing Entity

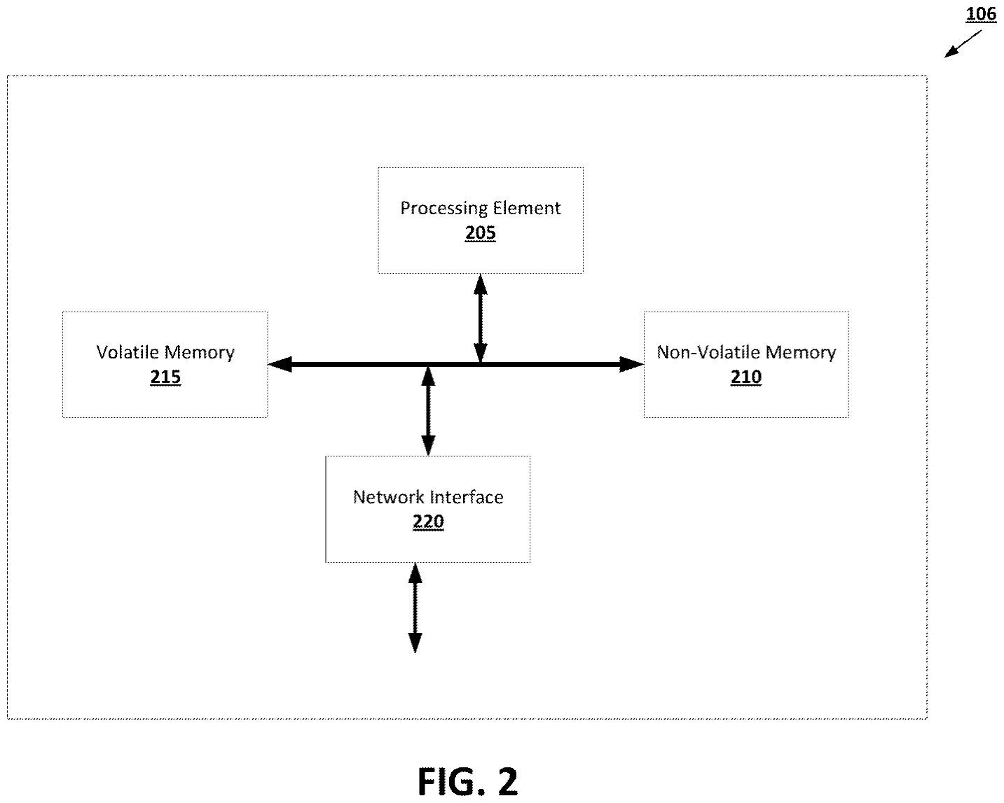

FIG. 2 provides a schematic of a predictive data analysis computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the predictive data analysis computing entity 106 may include, or be in communication with, one or more processing elements 205 (also referred to as processors, processing circuitry, and/or similar terms used herein interchangeably) that communicate with other elements within the predictive data analysis computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the predictive data analysis computing entity 106 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity-relationship model, object model, document model, semantic model, graph model, and/or the like.

In one embodiment, the predictive data analysis computing entity 106 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the predictive data analysis computing entity 106 with the assistance of the processing element 205 and operating system.

As indicated, in one embodiment, the predictive data analysis computing entity 106 may also include one or more communications interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the predictive data analysis computing entity 106 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data Rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the predictive data analysis computing entity 106 may include, or be in communication with, one or more input elements, such as a keyboard input, a mouse input, a touch screen/display input, motion input, movement input, audio input, pointing device input, joystick input, keypad input, and/or the like. The predictive data analysis computing entity 106 may also include, or be in communication with, one or more output elements (not shown), such as audio output, video output, screen/display output, motion output, movement output, and/or the like.

### Exemplary Client Computing Entity

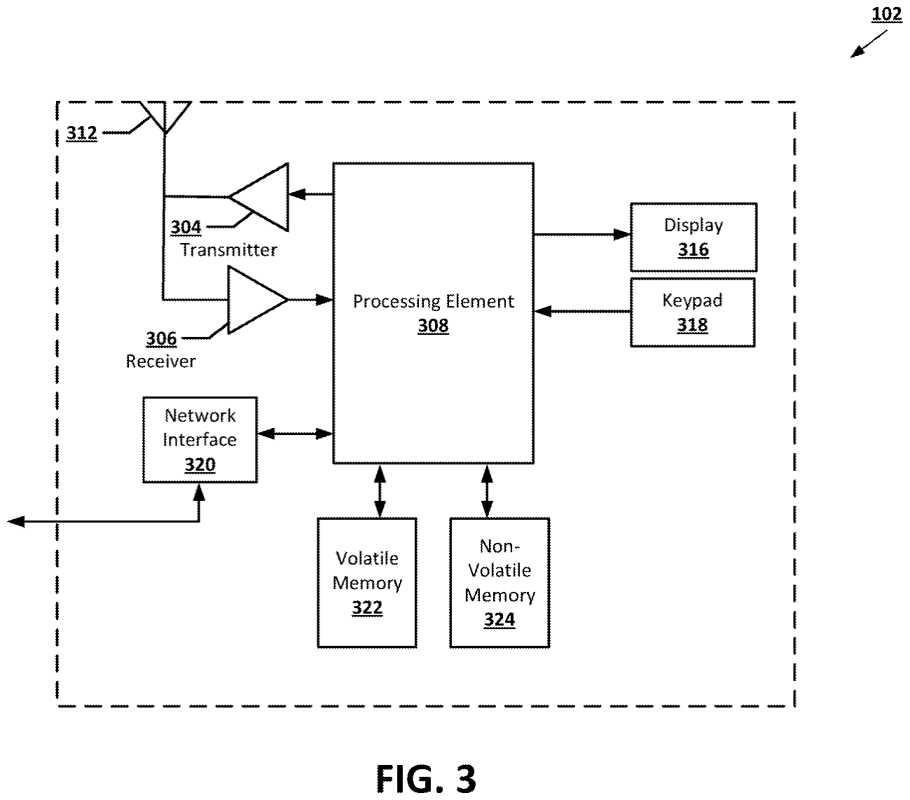

FIG. 3 provides an illustrative schematic representative of a client computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the predictive data analysis computing entity 106. In a particular embodiment, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the predictive data analysis computing entity 106 via a network interface 320.

Via these communication standards and protocols, the client computing entity 102 can communicate with various other entities using concepts such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the client computing entity 102 to interact with and/or cause display of information/data from the predictive data analysis computing entity 106, as described herein. The user input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the predictive data analysis computing entity 106 and/or various other computing entities.

In another embodiment, the client computing entity 102 may include one or more components or functionality that are the same or similar to those of the predictive data analysis computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Exemplary System Operations

Provided below are exemplary techniques for performing viral genomic predictive data analysis and for performing bacterial genomic predictive data analysis. However, while various embodiments of the present invention describe the viral genomic predictive data analysis operations described herein and the bacterial genomic predictive data analysis operations described herein as being performed by the same single computing entity, a person of ordinary skill in the relevant technology will recognize that each of the noted sets of operations described herein can be performed by one or more computing entities that may be the same as or different from the one or more computing entities used to perform each of the other sets of operations described herein.

As described below, various embodiments of the present invention improve efficiency of performing genomic predictive data analysis operations by performing feature extraction on selected defined-length subsequences of genomic sequences known as k-mers. For example, in one embodiment, a proposed solution determines, using the processor and a frequency-based k-mer extraction layer of a viral genome processing machine learning model, and based at least in part on the first genomic sequence, one or more frequent k-mers of the first genomic sequence; and then determines, using the processor and a one-dimensional convolutional neural network layer of the viral genome processing machine learning model, and based at least in part on the frequency-based refined representation, a viral replication origin k-mer of the one or more frequent k-mers. Performing genomic predictive data analysis operations based at least in part on k-mer features replaces the need for performing complex and computationally expensive feature extraction operations on typically long and character-intensive genomic sequences with less complex operations needed to detect selected k-mers such as frequent k-mers. In this way, various embodiments of the present invention reduce the number of processing operations needed to perform genomic predictive data analysis operations, make important technical contributions to improving efficiency of performing genomic predictive data analysis operations, and substantially improve the field of genomic predictive data analysis.

### Viral Genomic Predictive Data Analysis Operations

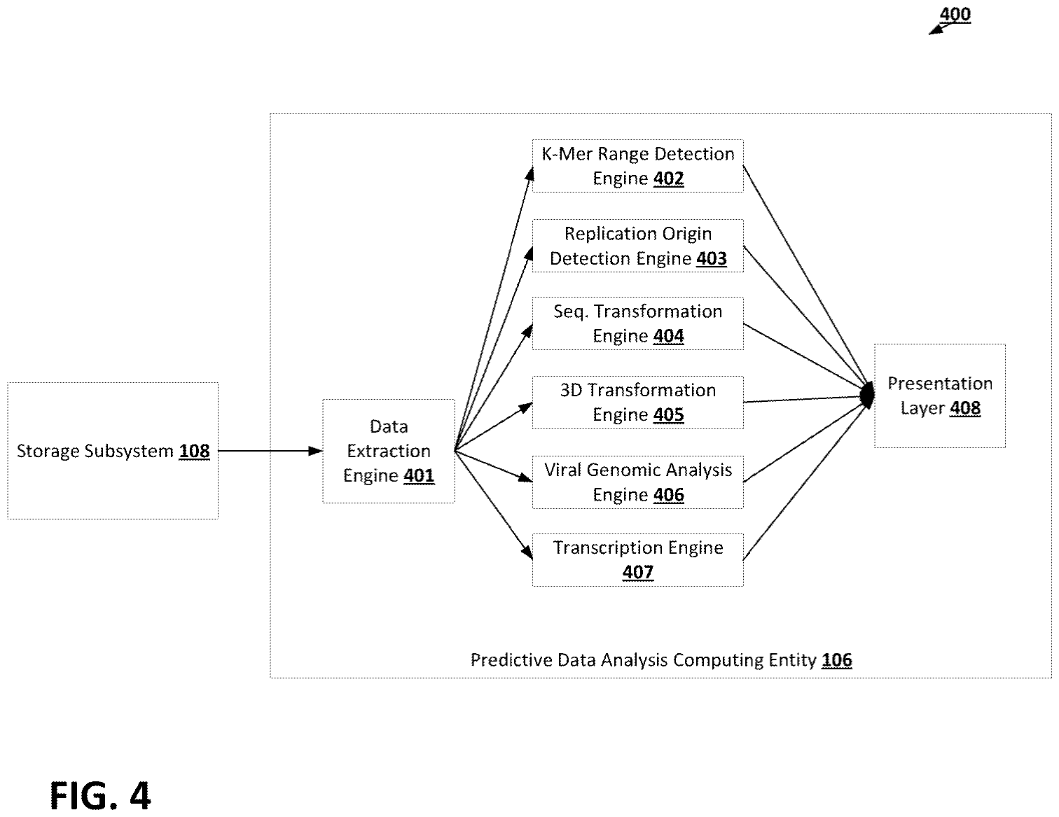

FIG. 4 is a data flow diagram of an example process 400 for performing viral genomic predictive data analysis on a genomic sequence set (i.e., a set of one or more genomic sequences). The process 400 enables the predictive data analysis computing entity 106 to perform a set of virus-related predictive genomic analysis inferences with respect to a genomic sequence set and perform a set of prediction-based actions based at least in part on the output of the set of predictive genomic analysis inferences.

The process 400 begins when a data extraction engine 401 of the predictive data analysis computing entity 106 extracts the genomic sequence set from the genomic sequence data that is stored on the storage subsystem 108 of the predictive data analysis system 101. In some embodiments, the genomic sequence data is received/retrieved/extracted from one or more genomic sequence databases, such as the National Center for Biotechnology Information (NCBI) genomic sequence database, the European Molecular Biology Laboratory (EMBL) genomic sequence database, DNA Data Bank of Japan (DDBJ) genomic sequence database, Protein Data Bank (PDB) genomic sequence database, Optum® genomic sequence database, and/or the like. In some embodiments, the genomic sequence set that is provided as an input for performing viral genomic predictive data analysis comprises genomic sequences that are detected to have viral infections. In some embodiments, the genomic sequence set that is provided as an input for performing viral genomic predictive data analysis comprises genomic sequences that are suspected of having viral infections.

The process 400 continues when the data extraction engine 401 provides the genomic sequence set to a k-mer range detection engine 402 of the predictive data analysis computing entity 106 that is configured to determine, for each genomic sequence in the genomic sequence set, an optimal k-mer range for the genomic sequence. The optimal k-mer range for a genomic sequence may describe a measure of an expected size of a subsequence of the genomic sequence that is deemed to be a viral replication origin for a viral infection that is associated with the genomic sequence. For example, the optimal k-mer range for a particular genomic sequence may describe that the viral replication origin for the particular genomic sequence may have a size of 100, such that the viral replication origin will be a 100-length character subsequence of the particular genomic sequence. In some embodiments, each viral infection is associated with an optimal k-mer range, and thus each genomic sequence that is associated with the noted viral infection will have the optimal k-mer range of the noted viral infection. In some embodiments, a character of a genomic sequence describes a genomic base occurrence within a particular location of the genomic sequence.

The process 400 continues when the data extraction engine 401 provides the genomic sequence set to a replication origin detection engine 403 of the predictive data analysis computing entity 106 that is configured to determine, for each genomic sequence, a viral replication origin k-mer. In some embodiments, to detect the viral replication origin k-mer for a genomic sequence, the replication origin detection engine 403: (i) identifies one or more frequent k-mers of the genomic sequence, and (ii) processes each frequent k-mer using a one-dimensional convolutional neural network machine learning model in order to determine whether the frequent k-mer is the viral replication origin k-mer.

In some embodiments, determining the viral replication origin k-mer for a genomic sequence comprises processing the genomic sequence (e.g., processing a one-hot-coded representation of the genomic sequence) using a viral genome processing machine learning model. A viral genome processing machine learning model may be a machine learning model that is configured to process a genomic sequence in order to generate a predictive output for the genomic sequence based at least in part on one or more k-mers of the genomic sequence. In some embodiments, the viral genome processing machine learning model comprises at least a k-mer extraction layer that is configured to determine one or more k-mers of a genomic sequence (e.g., one or more frequent k-mers of the genomic sequence) and a one-dimensional convolutional neural network layer that is configured to process the one or more k-mers to determine a predictive output for the genomic sequence (e.g., a viral replication origin k-mer for the genomic sequence).

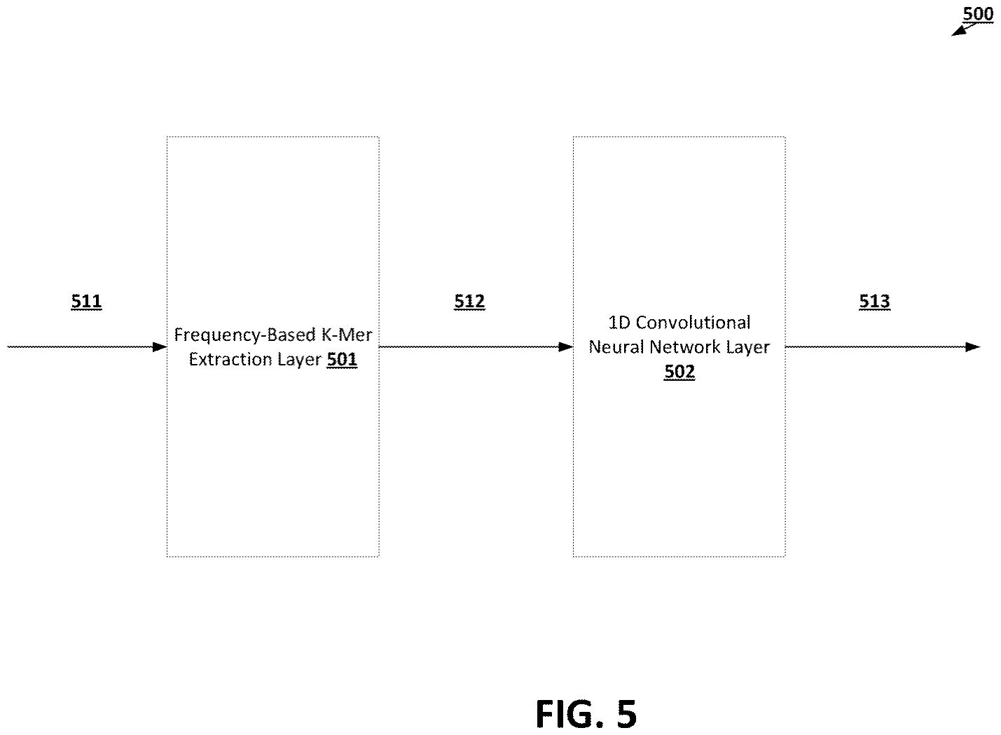

An operational example of a viral genome processing machine learning model 500 is depicted in FIG. 5. As depicted in FIG. 5, the viral genome processing machine learning model 500 comprises: (i) a frequency-based k-mer extraction layer 501 that is configured to determine one or more frequent k-mers 512 of an input genomic sequence 511, and (ii) a one-dimensional convolutional neural network layer 502 that is configured to process each frequent k-mer to determine a viral replication origin k-mer 513 of the frequent k-mers detected by the frequency-based k-mer extraction layer 501. Exemplary features of a frequency-based k-mer extraction layer and a one-dimensional convolutional neural network layer of a viral genome processing machine learning model are described in greater detail below.

In some embodiments, the frequency-based k-mer extraction layer of a viral genome processing machine learning model is configured to process a genomic sequence to determine one or more frequent k-mers of the genomic sequence. In some embodiments, a k-mer of a genomic sequence is any subsequence of the genomic subsequence that has a character length of k, where k may be determined by an optimal k-mer range for the genomic subsequence. In some embodiments, a frequent k-mer is a k-mer whose occurrence frequency score satisfies (e.g., exceeds) an occurrence frequency score threshold, where the occurrence frequency score of a k-mer with respect to a genomic sequence is detected based at least in part on a count of occurrence of the k-mer within the character string described by the noted genomic sequence. In some embodiments, the occurrence frequency score threshold that is used to classify a detected k-mer as a frequent k-mer or as an infrequent k-mer is determined based at least in part on an occurrence frequency score of a hypothesis k-mer that is determined to be a likely viral replication origin k-mer based at least in part on preexisting data about the virus at issue.

In some embodiments, determining the one or more frequent k-mers of a particular first genomic sequence comprises determining one or more detected k-mers of the particular first genomic sequence; for each detected k-mer, determining an occurrence frequency score within the particular first genomic sequence; and determining the one or more frequent k-mers based at least in part on each occurrence frequency score. In some embodiments, determining the one or more frequent k-mers based at least in part on each occurrence frequency score comprises: identifying a hypothesis k-mer of the one or more frequent k-mers; and for each detected k-mer, determining that the detected k-mer is one of the one or more frequent k-mers if the occurrence frequency score for the detected k-mer exceeds the occurrence frequency score for the hypothesis k-mer.

In some embodiments, the frequency-based k-mer extraction layer is configured to process a genomic sequence using a frequent words algorithm to determine one or more frequent k-mers of the genomic sequence. In some embodiments, the frequency-based k-mer extraction layer is configured to: (i) identify (e.g., using a sliding window of size k) one or more k¬-length subsequences of a genomic sequence, (ii) for each k¬-length subsequence, determine an occurrence frequency score, and (iii) determine one or more frequent k-mers of the genomic sequence based at least in part on each occurrence frequency score for a k¬-length subsequence. In some embodiments, the frequency-based k-mer extraction layer is configured to: (i) identify (e.g., using a sliding window of size k) one or more k¬-length subsequences of a genomic sequence, and (ii) determine that all of the one or more k¬-length subsequences are frequent k-mers of the genomic sequence (this may happen, for example, if the occurrence frequency score threshold is set to zero and a detected k-mer is deemed to be a frequent k-mer if the occurrence frequency score for the detected k-mer exceeds the noted occurrence frequency score threshold of zero).

In some embodiments, the one-dimensional convolutional layer of a viral genome processing machine learning model is configured to process an input k-mer (e.g., an input frequent k-mer) in order to determine an output value that describes a likelihood that the input k-mer is a viral replication origin k-mer for a genomic sequence that is associated with the noted input k-mer. In some embodiments, a frequent k-mer of a genomic sequence is determined to be the viral replication origin k-mer if the output likelihood that is generated for the frequent k-mer by the one-dimensional convolutional layer is higher than the output likelihoods generated by the one-dimensional convolutional layer for other frequent k-mers of the noted genomic sequence. For example, if a genomic sequence is associated with three frequent k-mers k₁, k₂, and k₃ that are respectively associated with output likelihood values p₁, p₂, and p₃, if p₁>p₂>p₃, then k₁ may be selected as the viral replication origin k-mer for the genomic sequence.

In some embodiments, the one-dimensional convolutional layer of a viral genome processing machine learning model is associated with a stride value and/or a kernel size. In some embodiments, the stride value and/or the kernel size of a one-dimensional convolutional layer defines the character length of input k-mers that may be provided as inputs to the one-dimensional convolutional layer. For example, in some embodiments, processing a frequent k-mer using a one-dimensional convolutional layer first comprises selecting the one-dimensional convolutional layer from a set of candidate one-dimensional convolutional layers each associated with a stride value and/or kernel size, where the selection of the one-dimensional convolutional layer comprises selecting a candidate one-dimensional convolutional layer whose stride value and/or kernel size is a defined ratio of the character length of the frequent k-mer. For example, in an exemplary embodiment, if the character length of a frequent k-mer is 100, then the one-dimensional convolutional layer should have a kernel size of [18, 22].

The viral replication origin k-mer of a genomic sequence may thus be a detected/frequent k-mer of the genomic sequence that is determined to be the most likely source of a viral infection that is associated with the noted genomic sequence. In some embodiments, the viral replication origin of a genomic sequence is a frequent k-mer of the genomic sequence that has a highest likelihood output as generated by the one-dimensional convolutional layer of a viral genome processing machine learning model. In some embodiments, if two or more frequent k-mers of a genomic sequence have an equal output likelihood as generated by the one-dimensional convolutional layer of a viral genome processing machine learning model, then the viral replication origin of a genomic sequence may be selected from the noted two or more frequent k-mers based at least in part on defined selection criteria (for example by selecting the frequent k-mers of the noted two or more frequent k-mers that has a lower character length). In some embodiments, if two or more frequent k-mers of a genomic sequence have an equal output likelihood as generated by the one-dimensional convolutional layer of a viral genome processing machine learning model, then a combination of the noted two or more frequent k-mers is deemed to be viral replication origins of the corresponding genomic sequence.

Accordingly, in some embodiments, the viral genome processing machine learning models described herein improve efficiency of performing genomic predictive data analysis operations by performing feature extraction on selected defined-length subsequences of genomic sequences known as k-mers. For example, in one embodiment, a proposed solution determines, using the processor and a frequency-based k-mer extraction layer of a viral genome processing machine learning model, and based at least in part on the first genomic sequence, one or more frequent k-mers of the first genomic sequence; and then determines, using the processor and a one-dimensional convolutional neural network layer of the viral genome processing machine learning model, and based at least in part on the frequency-based refined representation, a viral replication origin k-mer of the one or more frequent k-mers. Performing genomic predictive data analysis operations based at least in part on k-mer features replaces the need for performing complex and computationally expensive feature extraction operations on typically long and character-intensive genomic sequences with less complex operations needed to detect selected k-mers such as frequent k-mers. In this way, various embodiments of the present invention reduce the number of processing operations needed to perform genomic predictive data analysis operations, make important technical contributions to improving efficiency of performing genomic predictive data analysis operations, and substantially improve the field of genomic predictive data analysis.

Returning to FIG. 4, the process 400 continues when the data extraction engine 401 provides the genomic sequence set to a transcription engine 407 of the predictive data analysis computing entity 106 that is configured to perform one or more transcription operations on the genomic sequence set. In some embodiments, the transcription engine 407 is configured to perform one or more transcription operations and/or one or more translation operations on the genomic sequence set. In some embodiments, the transcription engine 407 is configured to detect a protein end-product of a virus associated with a genomic sequence set based at least in part on the output of performing one or more transcription operations and/or one or more translation operations on the genomic sequence set.

The process 400 continues when the data extraction engine 401 provides the genomic sequence set to a sequence transformation engine 404 of the predictive data analysis computing entity 106 that is configured to convert the format of the genomic sequence set to a standardized format. For example, the sequence transformation engine 404 may be configured to convert each genomic sequence of the genomic sequence set to a standardized format. An example of a standardized format is a format defined by the Fast Healthcare Interoperability Resources (FHRI) integration protocols. In some embodiments, the sequence transformation engine 404 creates/updates a database Molecular Sequence resource on an FHIR server device. In some embodiments, the standardization operations performed by the sequence transformation engine 404 enable at least one of the following: (i) using the interoperable FHIR framework for standardizing an informational flow of the predictive data analysis computing entity 106, (ii) using/creating FHIR resources to make genomic data available across the global healthcare community, and (iii) promoting innovation by sharing findings securely for research and development purposes.

The process 400 continues when the data extraction engine 401 provides the genomic sequence set to a three-dimensional transformation engine 405 of the predictive data analysis computing entity 106 that is configured to generate one or more three-dimensional views of the genomic sequence set. For example, the three-dimensional transformation engine 405 may be configured to generate one or more three-dimensional views for each genomic sequence of the genomic sequence set. In some embodiments, the three-dimensional views include a three-dimensional DNA model for the genomic sequence set and/or a three-dimensional DNA model for each genomic sequence of the genomic sequence set.

The process 400 continues when the data extraction engine 401 provides the genomic sequence set to a viral genomic analysis engine 406 that performs (e.g., using one or more integrated artificial intelligence and/or machine learning resources) one or more predictive genomic analysis operations on the genomic sequence set in order to generate one or more viral predictions for one or more viruses that are associated with the genomic sequence set. For example, the viral genomic analysis engine 406 may perform one or more predictive genomic analysis operations on each genomic sequence of the noted genomic sequence set in order to generate one or more viral predictions for the virus that is associated with the noted genomic sequence. Examples of predictive genomic analysis operations that may be performed on genomic sequence sets and/or on genomic sequences include phylogenetic analysis operations (e.g., to generate virulence and novel strains predictions) and/or reverse transcriptase gene prediction operations.

In some embodiments, at least some of the predictive genomic analysis operations that may be performed on a genomic sequence set are performed based at least in part on each viral replication origin k-mer for the genomic sequence in the noted genomic sequence set. In some embodiments, at least some of the predictive genomic analysis operations that may be performed on a genomic sequence are performed based at least in part on the viral replication origin k-mer for the genomic sequence. For example, in some embodiments, the viral genomic analysis engine 406 identifies a plurality of viral replication origin k-mers (e.g., including viral replication origin k-mers that were identified by a viral genome processing machine learning model) and determines one or more viral genome clusters based at least in part on the plurality of viral replication origin k-mers. In some of the noted embodiments, upon identifying (e.g., receiving) an input viral genomic sequence, the viral genomic analysis engine 406 determines a viral cluster similarity measure with respect to the input viral genomic sequence and determines a viral strain prediction for the input viral genomic sequence based at least in part on each viral cluster similarity measure.

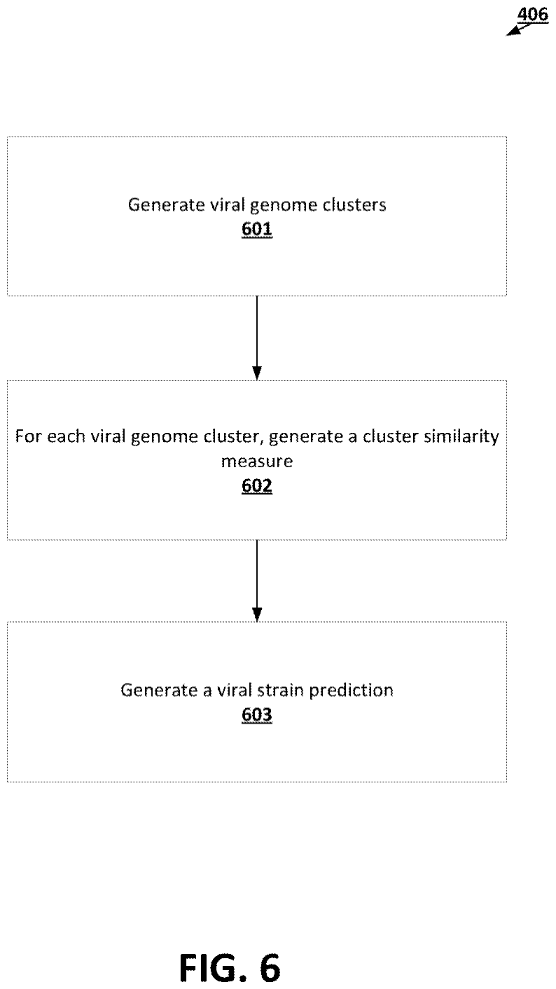

In some embodiments, performing the operations of the viral genomic analysis engine 406 comprises performing the steps/operations of FIG. 6, which is a flowchart diagram of an example process for generating a viral strain prediction for an input viral genomic sequence. The process that is depicted in FIG. 6 begins at step/operation 601 when the viral genomic analysis engine 406 generates one or more viral genome clusters based at least in part on a plurality of viral replication origin k-mers. In some embodiments, each viral genome cluster is a subset of a plurality viral replication origin k-mers that is deemed to have similar viral/genomic properties. In some embodiments, determining the viral genome clusters comprises: (i) mapping each viral replication origin k-mer to an n-dimensional value in an n-dimensional space, and (ii) performing one or more clustering operations (e.g., one or more k-means clustering operations, one or more k-nearest-neighbor clustering operations, and/or the like) on the n-dimensional space to generate the viral genome cluster.

Examples of features that may be associated with the n dimensions of the n-dimensional space comprise the number of adenine (A) characters in a k-mer, the number of thymine (T) characters in a k-mer, the number of guanine (G) characters in a k-mer, the number of cytosine (C) characters in a k-mer, the ratio of adenine (A) characters in a k-mer, the ratio of thymine (T) characters in a k-mer, the ratio of guanine (G) characters in a k-mer, the ratio of cytosine (C) characters in a k-mer, features describing the number and/or the ratio of combinations of the noted genomic base characters in a k-mer, features describing the number and/or the ratio of genomic base characters in a specified ratio of a k-mer (e.g., in a substring of the k-mer that includes the first two percent of the genomic base characters of the k-mer), features describing the number and/or the ratio of combinations of genomic base characters in a specified ratio of a k-mer (e.g., in a substring of the k-mer that includes the first two percent of the genomic base characters of the k-mer), and/or the like.

At step/operation 602, the viral genomic analysis engine 406 determines a viral cluster similarity measure for each viral genome cluster that describes a deviation measure for the input viral genomic sequence with respect to the viral genome cluster. For example, in some embodiments, to determine a viral cluster similarity measure for a viral genome cluster, the viral genomic analysis engine 406 may: (i) identify one or more selected k-mers (e.g., one or more defined k-mers) of the input viral genomic sequence, (ii) map each selected k-mers to the multi-dimensional space of the viral genome cluster, (iii) for each selected k-mer, determine a distance measure between the mapping of the k-mer in the multi-dimensional space and a defined point of the viral genome cluster in the multi-dimensional space (e.g., a defined centroid point of the viral genome cluster in the multi-dimensional space), and (iv) determine the viral cluster similarity measure based at least in part on each distance measure for a selected k-mer (e.g., based at least in part on a sum of each distance measure for a selected k-mer, based at least in part on a statistical distribution measure such as an average of each distance measure for a selected k-mer, and/or the like).

As another example, in some embodiments, to determine a viral cluster similarity measure for a viral genome cluster, the viral genomic analysis engine 406 may: (i) identify one or more selected k-mers (e.g., one or more defined k-mers) of the input viral genomic sequence, (ii) map each selected k-mers to the multi-dimensional space of the viral genome cluster, (iii) process each selected k-mer using a one-dimensional convolutional layer of a viral genome processing machine learning model to generate an output likelihood for the selected k-mer, (iv) for each selected k-mer, determine a distance measure between the mapping of the k-mer in the multi-dimensional space and a defined point of the viral genome cluster in the multi-dimensional space (e.g., a defined centroid point of the viral genome cluster in the multi-dimensional space), (v) for each selected k-mer, determine a weighted distance based at least in part on the distance measure for the selected k-mer and the generated output likelihood for the selected k-mer (e.g., based at least in part on the output of applying the distance measure for the selected k-mer to the generated output likelihood for the selected k-mer), and (iv) determine the viral cluster similarity measure based at least in part on each weighted distance measure for a selected k-mer (e.g., based at least in part on a sum of each weighted distance measure for a selected k-mer, based at least in part on a statistical distribution measure such as an average of each weighted distance measure for a selected k-mer, and/or the like).

At step/operation 603, the viral genomic analysis engine 406 generates a viral strain prediction for the input viral genomic sequence based at least in part on each viral cluster similarity measure for a viral genome cluster. For example, in some embodiments, the viral genomic analysis engine 406 may determine that the input viral genomic sequence has a viral strain prediction defined by one or more viral/genomic properties of a viral genome cluster having a highest viral cluster similarity measure among the viral cluster similarity measures of the plurality of viral cluster similarity measures. As another example, in some embodiments, the viral genomic analysis engine 406 may determine that the input viral genomic sequence has a viral strain prediction defined by one or more viral/genomic properties of each viral genome cluster having a threshold-satisfying viral cluster similarity measure.

Returning to FIG. 4, the outputs of the k-mer range detection engine 402, the replication origin detection engine 403, the sequence transformation engine 404, the three-dimensional transformation engine 405, the viral genomic analysis engine 406, and/or the transcription engine 407 may be used to perform one or more prediction-based actions. For example, the viral genomic analysis engine 406 may be configured to detect novel virus strains, which then may be used to perform one or more operational load balancing operations by generating notifications for hospital staff to be prepared for particular operational requirements, by allocating hospital equipment in accordance with expected surges in need for medical services due to novel viral strains, by generating automated medical appointments for those patients that are deemed to be at a higher risk for novel viral strains because of genetic factors detected based at least in part on the patients' genomic sequencing data, and/or the like.

In some embodiments, performing the prediction-based actions includes generating one or more prediction output user interfaces that enable an end user to access the outputs of the k-mer range detection engine 402, the replication origin detection engine 403, the sequence transformation engine 404, the three-dimensional transformation engine 405, the viral genomic analysis engine 406, and/or the transcription engine 407. For example, as depicted in FIG. 4, the process 400 includes using a presentation layer 408 to generate user interface data for one or more prediction-output user interfaces to display outputs of the k-mer range detection engine 402, the replication origin detection engine 403, the sequence transformation engine 404, the three-dimensional transformation engine 405, the viral genomic analysis engine 406, and/or the transcription engine 407. For example, the prediction output user interfaces may include an OriView user interface dashboard to display the viral replication origin k-mer for a set of input genomic sequences, a GenomeView user interface dashboard to display viral strain predictions for a set of input viral genomic sequence, an FHIRView user interface dashboard to display standardization output for a set of input viral genomic sequence, a 3DView user interface dashboard to display three-dimensional graphical representations of a set of input viral genomic sequence, and/or the like. In some embodiments, the user interface data for prediction output user interfaces are transmitted to the client computing entities 102 for display by display devices of the client computing entities 102 to end users of the client computing entities 102.

### Bacterial Genomic Predictive Data Analysis Operations

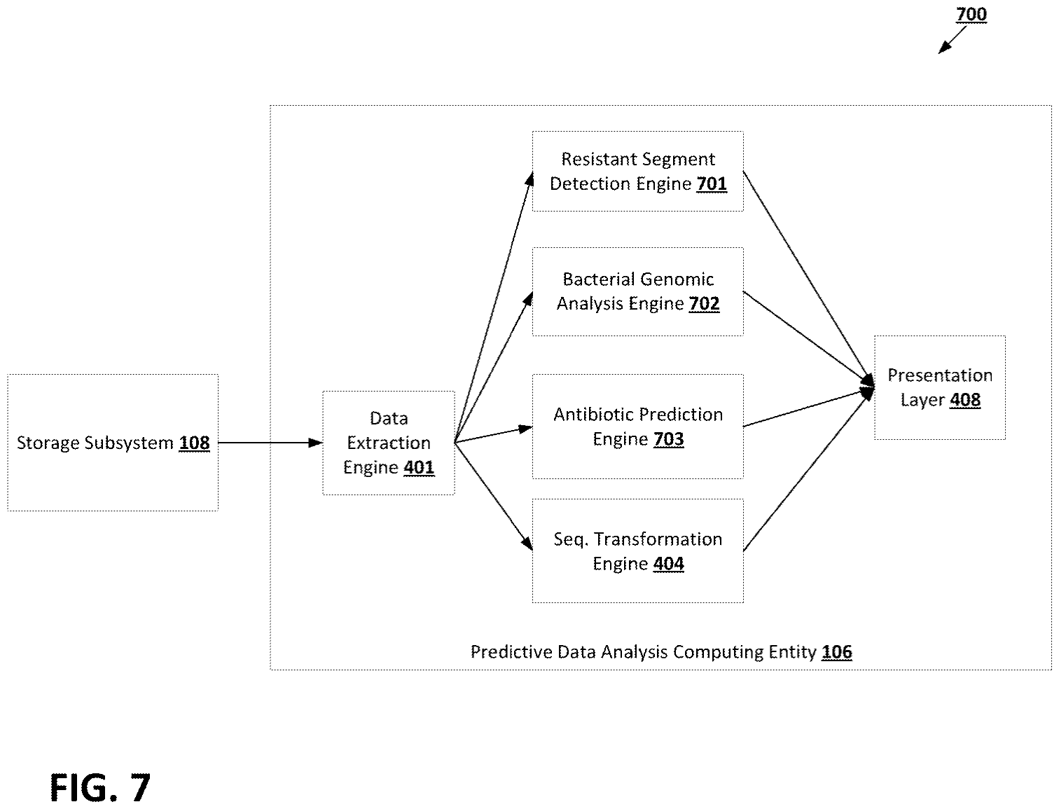

FIG. 7 is a data flow diagram of an example process 700 for performing bacterial predictive genomic analysis on a genomic sequence set (i.e., a set of one or more genomic sequences). The process 700 enables the predictive data analysis computing entity 106 to perform a set of bacteria-related predictive genomic analysis inferences with respect to a genomic sequence set and perform a set of prediction-based actions based at least in part on the output of the set of predictive genomic analysis inferences.

The process 700 begins when a data extraction engine 401 of the predictive data analysis computing entity 106 extracts the genomic sequence set from the genomic sequence data that is stored on the storage subsystem 108 of the predictive data analysis system 101. In some embodiments, the genomic sequence data is received/retrieved/extracted from one or more genomic sequence databases, such as the National Center for Biotechnology Information (NCBI) genomic sequence database, the European Molecular Biology Laboratory (EMBL) genomic sequence database, DNA Data Bank of Japan (DDBJ) genomic sequence database, Protein Data Bank (PDB) genomic sequence database, Optum® genomic sequence database, and/or the like. In some embodiments, the genomic sequence set that is provided as an input for performing viral genomic predictive data analysis comprises genomic sequences that are detected to have viral infections. In some embodiments, the genomic sequence set that is provided as an input for performing bacterial genomic analysis predictive comprises genomic sequences that are suspected of having bacterial infections.

The process 700 continues when the data extraction engine 401 provides the genomic sequence set to a resistant segment detection engine 701 of the predictive data analysis computing entity 106 that is configured to determine one or more resistant bacterial segments of the genomic sequence set. In some embodiments, to determine the one or more resistant bacterial segments of a genomic sequence set, the resistant segment detection engine 701 processes the genomic sequence set (e.g., a one-hot-coded representation of the genomic sequence set, which may be a two-dimensional matrix) using a bacterial genome processing machine learning model to generate the one or more resistant bacterial segments of the second genomic sequence set.

In some embodiments, the genome processing machine learning model is configured to apply a two-dimensional convolution to a two-dimensional representation of genomic sequence set (e.g., a two-dimensional data object including each one-hot-coded representation of a genomic sequence of the genomic sequence set) to determine each segment of the two-dimensional representation that is likely to be associated with a resistant bacterial segment. In some embodiments, in addition to and/or instead of using the two-dimensional convolutional operations, the genome processing machine learning model is configured to process the genomic sequence set using other pattern matching operations in order to determine each segment of the genomic sequence set that is likely to be associated with a resistant bacterial segment.

A resistant bacterial segment may be a segment of a genomic sequence set that is determined to be resistant to a set of one or more antibacterial medications. In some embodiments, a bacterial genome processing machine learning model processes a genomic sequence set to determine one or more resistant bacterial segments of the genomic sequence set. In some embodiments, a resistant bacterial segment is a two-dimensional subset of a genomic sequence. In some embodiments, to determine a resistant bacterial segment for a genomic sequence, a bacterial genome processing machine learning model utilizes a two-dimensional convolutional neural network layer, such as a two-dimensional convolutional neural network layer whose stride value determined based at least in part on a defined ratio of a number of elements of the genomic sequence set, or a two-dimensional convolutional neural network layer whose kernel dimension lengths are determined based at least in part on a defined ratio of the dimension lengths of the two dimensions of the genomic sequence. In some embodiments, the bacterial genome processing machine learning model is further configured to determine a non-resistant antibacterial recommendation for each resistant bacterial segment that describes an antibacterial medication to which the resistant bacterial segment is deemed to be non-resistant. In some embodiments, for each non-resistant antibacterial recommendation for a genomic sequence set, the bacterial genome processing machine learning model generates a recommendation score that describes an inferred/computed likelihood that the noted genomic sequence is non-resistant to an antibacterial medication of the non-resistant antibacterial recommendation.

The process 700 continues when the data extraction engine 401 provides the genomic sequence set to a bacterial genomic analysis engine 702 of the predictive data analysis computing entity 106 that is configured to perform (e.g., using one or more integrated artificial intelligence and/or machine learning resources) one or more predictive genomic analysis operations on the genomic sequence set in order to generate one or more bacterial predictions for one or more bacterial infections that are associated with the genomic sequence set. Examples of predictive genomic analysis operations that may be performed on genomic sequence sets and/or on genomic sequences include clinical decision support system operations, antibiotic efficacy computation operation, antibiotic resistance surveillance operations, and/or the like.

In some embodiments, the predictive genomic analysis operations identify a location-wise frequency measure for each resistant bacterial segment within genomic sequence data for a location-wide designation of the second genomic sequence set and generate a location-wise bacterial spread prediction for the location-wide designation based at least in part on each location-wise frequency measure. The location-wise frequency measure may describe an occurrence frequency and/or an occurrence frequency ratio of a particular antibacterial resistance that is associated with a resistant bacterial segment within a defined locality (e.g., a state, a county, a country, and/or the like). For example, in some embodiments, high occurrence frequency of resistance to penicillin within New York City may be used to generate a location-wise bacterial spread prediction describing the inferred pattern for the location-wide designation of New York City. The noted inferred pattern may be determined based at least in part on resistant bacterial segments that were detected for monitored individuals that reside in the noted locality.

In some embodiments, the predictive genomic analysis operations identify a demographic frequency measure for each resistant bacterial segment within genomic sequence data for a demographic designation of the second genomic sequence set and generate a demographic bacterial spread prediction for the demographic designation based at least in part on each demographic frequency measure. The location-wise frequency measure may describe an occurrence frequency and/or an occurrence frequency ratio of a particular antibacterial resistance that is associated with a resistant bacterial segment within a defined demographic (e.g., 18-22 year old Hispanic men who live in the State of New York). For example, in some embodiments, high occurrence frequency of resistance to penicillin within 18-22 year old Hispanic men who live in the State of New York may be used to generate a demographic bacterial spread prediction describing the inferred pattern for the demographic designation that is associated with 18-22 year old Hispanic men who live in the State of New York. The noted inferred pattern may be determined based at least in part on resistant bacterial segments that were detected for monitored individuals that reside in the noted demographic group.

The process 700 continues when the data extraction engine 401 provides the genomic sequence set to an antibiotic prediction engine 703 of the predictive data analysis computing entity 106 that is configured to determine, for each resistant bacterial segment, a non-resistant antibacterial recommendation describing one or more antibacterial medications to which the resistant bacterial segment is not resistant. In some embodiments, the non-resistant antibacterial recommendation for a resistant bacterial segment is an output of the bacterial genome processing machine learning model that generates/identifies the resistant bacterial segment. For example, in some embodiments, the bacterial genome processing machine learning model comprises a two-dimensional convolutional neural network layer that is configured to determine one or more resistant bacterial segments of a genome sequence and determine a non-resistant antibacterial recommendation for each resistant bacterial segment. In some embodiments, the non-resistant antibacterial recommendation for a resistant bacterial segment describes a recommendation score for each recommended antibacterial medication, where the recommendation score may also be the output of the bacterial genome processing machine learning model that generates/identifies the resistant bacterial segment. For example, in some embodiments, the bacterial genome processing machine learning model comprises a two-dimensional convolutional neural network layer that is configured to determine one or more resistant bacterial segments of a genome sequence and determine, for each resistant bacterial segment, a recommendation score for each antibacterial medication of a set of candidate antibacterial medications with respect to the resistant bacterial segment.

The process 700 continues when the data extraction engine 401 provides the genomic sequence set to the sequence transformation engine 404 of the predictive data analysis computing entity 106 that is configured to convert the format of the genomic sequence set to a standardized format. For example, the sequence transformation engine 404 may be configured to convert each genomic sequence of the genomic sequence set to a standardized format. An example of a standardized format is a format defined by the Fast Healthcare Interoperability Resources (FHRI) integration protocols. In some embodiments, the sequence transformation engine 404 creates/updates a database Molecular Sequence resource on an FHIR server device. In some embodiments, the standardization operations performed by the sequence transformation engine 404 enable at least one of the following: (i) using the interoperable FHIR framework for standardizing an informational flow of the predictive data analysis computing entity 106, (ii) using/creating FHIR resources to make genomic data available across the global healthcare community, and (iii) promoting innovation by sharing findings securely for research and development purposes.

The outputs of the resistant segment detection engine 701, the bacterial genomic analysis engine 702, and/or the sequence transformation engine 404 may be used to perform one or more prediction-based actions. For example, the viral genomic analysis engine 406 may be configured to detect prevalent bacterial infections, which then may be used to perform one or more operational load balancing operations by generating notifications for hospital staff to be prepared for particular operational requirements, by allocating hospital equipment in accordance with expected surges in need for medical services due to prevalent bacterial infections, by generating automated medical appointments for those patients that are deemed to be at a higher risk for prevalent bacterial infections because of genetic factors detected based at least in part on the patients' genomic sequencing data, and/or the like.

In some embodiments, performing the prediction-based actions includes generating one or more prediction output user interfaces that enable an end user to access the viral genomic analysis engine 406 the outputs of the outputs of the resistant segment detection engine 701, the bacterial genomic analysis engine 702, and/or the sequence transformation engine 404. For example, as depicted in FIG. 7, the process 700 includes using a presentation layer 408 to generate user interface data for one or more prediction-output user interfaces to display outputs of the resistant segment detection engine 701, the bacterial genomic analysis engine 702, and/or the sequence transformation engine 404. For example, the prediction output user interfaces may include display resistant bacterial segments for genomic sequence sets, antibacterial recommendation scores for genomic sequences, at-risk localities and/or demographic groups for particular bacterial resistance conditions, and/or the like. In some embodiments, the user interface data for prediction output user interfaces are transmitted to the client computing entities 102 for display by display devices of the client computing entities 102 to end users of the client computing entities 102.

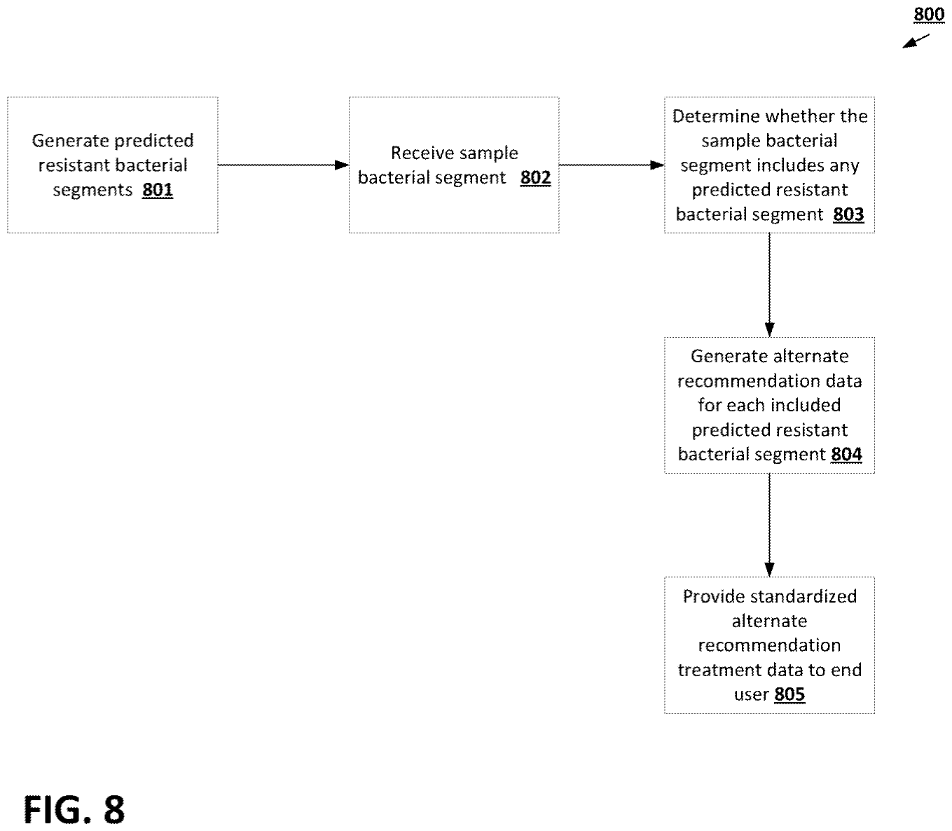

In some embodiments, performing the prediction-based actions based at least in part on a set of predicted resistant bacterial segments may be performed in accordance with the process 800 that is depicted in FIG. 8. The process 800 that is depicted in FIG. 8 includes generating the set of predicted resistant bacterial segments at step/operation 801 and receiving a sample bacterial genome segment at step/operation 802 by the predictive data analysis computing entity 106. In some embodiments, the predictive data analysis computing entity 106 enables access to a bacterial genome processing application programming interface (API) that is configured to receive the sample bacterial genome segment as part of an API call for the bacterial genome processing API.

At step/operation 803, the predictive data analysis determines whether the sample bacterial genome segment includes any of the predicted resistant bacterial segments. At step/operation 804, for each predicted resistant bacterial segment that is found within the sample bacterial segment, alternate treatment recommendation data (e.g., one or more non-resistant antibacterial medications) is generated. At step/operation 805, the alternate treatment recommendation data is standardized in accordance with FHIR protocols and provided to a client computing entity 102 in response to an API call by the client computing entity 102, e.g., as part of an API call response for the bacterial genome processing API.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for performing predictive genomic analysis, the computer-implemented method comprising:
identifying, using a processor, a first genomic sequence set;
for each first genomic sequence in the first genomic sequence set:
determining, using the processor and a frequency-based k-mer extraction layer of a viral genome processing machine learning model, and based at least in part on the first genomic sequence, one or more frequent k-mers of the first genomic sequence; and
determining, using the processor and a one-dimensional convolutional neural network layer of the viral genome processing machine learning model, and based at least in part on the frequency-based refined representation, a viral replication origin k-mer of the one or more frequent k-mers; and
performing, using the processor, one or more prediction-based actions based at least in part on each viral replication origin k-mer.

2. The computer-implemented method of claim 1, further comprising:
identifying, using the processor, a plurality of viral replication origin k-mers, wherein the plurality of viral replication origin k-mers comprise each viral replication origin k-mer for the first genomic sequence set; and
determining, using the processor, one or more viral genome clusters based at least in part on the plurality of viral replication origin k-mers.

3. The computer-implemented method of claim 2, further comprising:
identifying, using the processor, an input viral genomic sequence;
for each viral genome clusters, determining, using the processor, a viral cluster similarity measure with respect to the input viral genomic sequence; and
determining, using the processor, a viral strain prediction for the input viral genomic sequence based at least in part on each viral cluster similarity measure.

4. The computer-implemented method of claim 1, wherein determining the one or more frequent k-mers of a particular first genomic sequence comprises:
determining one or more detected k-mers of the particular first genomic sequence;
for each detected k-mer, determining an occurrence frequency score within the particular first genomic sequence; and
determining the one or more frequent k-mers based at least in part on each occurrence frequency score.

5. The computer-implemented method of claim 4, wherein determining the one or more frequent k-mers based at least in part on each occurrence frequency score comprises:
identifying a hypothesis k-mer of the one or more frequent k-mers; and
for each detected k-mer, determining that the detected k-mer is one of the one or more frequent k-mers if the occurrence frequency score for the detected k-mer exceeds the occurrence frequency score for the hypothesis k-mer.

6. The computer-implemented method of claim 1, further comprising:
identifying, using the processor, a second genomic sequence set;
determining, using the processor and a two-dimensional convolutional neural network layer of a bacterial genome processing machine learning model, and based at least in part on the second genomic sequence set, one or more resistant bacterial segments of the second genomic sequence set; and
performing, using the processor, one or more second prediction-based actions based at least in part on the one or more resistant bacterial segments.

7. The computer-implemented method of claim 6, wherein the two-dimensional convolutional neural network layer is further configured to determine a non-resistant antibacterial recommendation for each resistant bacterial segment.

8. The computer-implemented method of claim 6, further comprising:
for each resistant bacterial segment, identifying, using the processor, a location-wise frequency measure for the resistant bacterial segment within genomic sequence data for a location-wide designation of the second genomic sequence set; and
generating, using the processor, a location-wise bacterial spread prediction for the location-wide designation based at least in part on each location-wise frequency measure.

9. The computer-implemented method of claim 6, further comprising:
for each resistant bacterial segment, identifying, using the processor, a demographic frequency measure for the resistant bacterial segment within genomic sequence data for a location-wide designation of the second genomic sequence set;
generating, using the processor, a demographic bacterial spread prediction for the demographic designation based at least in part on each demographic frequency measure.

10. An apparatus for performing predictive genomic analysis, the apparatus comprising at least one processor and at least one memory including program code, the at least one memory and the program code configured to, with the processor, cause the apparatus to at least:
identify a first genomic sequence set;
for each first genomic sequence in the first genomic sequence set:
determine, using a frequency-based k-mer extraction layer of a viral genome processing machine learning model and based at least in part on the first genomic sequence, one or more frequent k-mers of the first genomic sequence; and
determine, using a one-dimensional convolutional neural network layer of the viral genome processing machine learning model and based at least in part on the frequency-based refined representation, a viral replication origin k-mer of the one or more frequent k-mers; and
perform one or more prediction-based actions based at least in part on each viral replication origin k-mer.

11. The apparatus of claim 10, wherein the at least one memory and the program code are further configured to, with the processor, cause the apparatus to at least:
identify a plurality of viral replication origin k-mers, wherein the plurality of viral replication origin k-mers comprise each viral replication origin k-mer for the first genomic sequence set; and
determine one or more viral genome clusters based at least in part on the plurality of viral replication origin k-mers.

12. The apparatus of claim 11, wherein the at least one memory and the program code are further configured to, with the processor, cause the apparatus to at least:
identify an input viral genomic sequence;
for each viral genome clusters, determine a viral cluster similarity measure with respect to the input viral genomic sequence; and
determine a viral strain prediction for the input viral genomic sequence based at least in part on each viral cluster similarity measure.

13. The apparatus of claim 10, wherein determining the one or more frequent k-mers of a particular first genomic sequence comprises:
determining one or more detected k-mers of the particular first genomic sequence;
for each detected k-mer, determining an occurrence frequency score within the particular first genomic sequence; and
determining the one or more frequent k-mers based at least in part on each occurrence frequency score.

14. The apparatus of claim 13, wherein determining the one or more frequent k-mers based at least in part on each occurrence frequency score comprises:
identifying a hypothesis k-mer of the one or more frequent k-mers; and
for each detected k-mer, determine that the detected k-mer is one of the one or more frequent k-mers if the occurrence frequency score for the detected k-mer exceeds the occurrence frequency score for the hypothesis k-mer.

15. The apparatus of claim 10, wherein the at least one memory and the program code are further configured to, with the processor, cause the apparatus to at least:
identify a second genomic sequence set;
determine, using a two-dimensional convolutional neural network layer of a bacterial genome processing machine learning model and based at least in part on the second genomic sequence set, one or more resistant bacterial segments of the second genomic sequence set; and
perform one or more second prediction-based actions based at least in part on the one or more resistant bacterial segments.

16. The apparatus of claim 15, wherein the two-dimensional convolutional neural network layer is further configured to determine a non-resistant antibacterial recommendation for each resistant bacterial segment.

17. The apparatus of claim 15, wherein the at least one memory and the program code are further configured to, with the processor, cause the apparatus to at least:
for each resistant bacterial segment, identify a location-wise frequency measure for the resistant bacterial segment within genomic sequence data for a location-wide designation of the second genomic sequence set; and
generate a location-wise bacterial spread prediction for the location-wide designation based at least in part on each location-wise frequency measure.

18. The apparatus of claim 15, wherein the at least one memory and the program code are further configured to, with the processor, cause the apparatus to at least:
for each resistant bacterial segment, identify a demographic frequency measure for the resistant bacterial segment within genomic sequence data for a location-wide designation of the second genomic sequence set;
generate a demographic bacterial spread prediction for the demographic designation based at least in part on each demographic frequency measure.

19. A computer program product for performing predictive genomic analysis, the computer program product comprising at least one non-transitory computer readable storage medium having computer-readable program code portions stored therein, the computer-readable program code portions configured to:
identify a first genomic sequence set;
for each first genomic sequence in the first genomic sequence set:
determine, using a frequency-based k-mer extraction layer of a viral genome processing machine learning model and based at least in part on the first genomic sequence, one or more frequent k-mers of the first genomic sequence; and
determine, using a one-dimensional convolutional neural network layer of the viral genome processing machine learning model and based at least in part on the frequency-based refined representation, a viral replication origin k-mer of the one or more frequent k-mers; and
perform one or more prediction-based actions based at least in part on each viral replication origin k-mer.

20. The computer program product of claim 19, wherein the computer-readable program code portions are further configured to:
identify a plurality of viral replication origin k-mers, wherein the plurality of viral replication origin k-mers comprise each viral replication origin k-mer for the first genomic sequence set; and
determine one or more viral genome clusters based at least in part on the plurality of viral replication origin k-mers.
